(19) United States
(12) Patent Application Publication
(10) Pub. No.: US 2018/0271445 A1
(43) Pub. Date: Sep. 27, 2018

(54) VASCULAR SENSOR IMPLANTATION DEVICES AND METHODS

(71) Applicant: ST. JUDE MEDICAL, CARDIOLOGY DIVISION, INC., St. Paul, MN (US)

(72) Inventors: Peter N. BRAIDO, Wyoming, MN (US); Neelakantan SAIKRISHNAN, Plymouth, MN (US); Mina S. FAHIM, Shoreview, MN (US)

(73) Assignee: ST. JUDE MEDICAL, CARDIOLOGY DIVISION, INC., St. Paul, MN (US)

(21) Appl. No.: 15/765,428

(22) PCT Filed: Oct. 12, 2016
(86) PCT No.: PCT/US2016/056552

§ 371 Date: Apr. 2, 2018

Related U.S. Application Data
(60) Provisional application No. 62/241,648, filed on Oct. 14, 2015.

(51) Int. Cl. A61B 5/00; A61B 5/07; A61B 5/0215; A61B 17/00

(52) U.S. Cl. CPC A61B 5/6876; A61B 5/076; A61B 5/0215; A61B 5/6882; A61B 5/6883; A61B 5/6884; A61B 2017/0464; A61B 2560/063; A61B 2562/028; A61B 2560/0219; A61B 2017/00004; A61B 2017/00867; A61B 17/0057

(57) ABSTRACT

A sensor implantation assembly is disclosed that includes a tissue puncture closure device. The tissue puncture closure device has a proximal end portion and a distal end portion and a suture extending from the proximal end portion to the distal end portion. A suture anchor assembly is also included that is insertable through a tissue wall puncture. The suture anchor assembly is attached to the suture at the distal end portion of the closure device and has a diagnostic sensor. A sealing pad is positioned around the suture at the distal end portion and is slidable along the suture. The diagnostic sensor may be placed within a tissue wall to gather diagnostic information such as pressure measurements at or near the site of the tissue puncture closure.

### CROSS REFERENCE TO RELATED APPLICATIONS

This application claims priority to U.S. Provisional Patent Application No. 62/241,648, filed Oct. 14, 2015, which is hereby incorporated by reference in its entirety.

### TECHNICAL FIELD

The present disclosure generally relates to medical devices and more particularly to devices for sealing punctures or incisions in a tissue wall and implanting sensors on or in the tissue wall.

### BACKGROUND

Various surgical procedures are routinely carried out intravascularly or intraluminally. For example, in the treatment of vascular disease, such as arteriosclerosis, it is a common practice to invade the artery and insert an instrument (e.g., a balloon or other type of catheter) to carry out a procedure within the artery. Such procedures usually involve the percutaneous puncture of the artery so that an insertion sheath can be placed in the artery and, thereafter, instruments (e.g., a catheter) can pass through the sheath and to an operative position within the artery. Intravascular and intraluminal procedures unavoidably present the problem of stopping the bleeding at the percutaneous puncture after the procedure has been completed and after the instruments (and any insertion sheaths used therewith) have been removed. Bleeding from puncture sites, particularly in the case of femoral arterial punctures, is typically stopped by utilizing vascular closure devices, such as those described in U.S. Pat. Nos. 6,090,130 and 6,045,569 and related patents that are hereby incorporated by reference in their entirety,

Furthermore, diagnostics are employed in nearly every aspect of medicine today, but it can often be expensive, obstrusive, unreliable, or not possible in underserved regions. Recent technological advances in micro electromechanical systems (MEMS) can however address many of the shortcomings in diagnostic care and improve clinical outcomes and costs. Implanted MEMS devices can measure real time pressure, flow, forces, cardiac output, orifice area, pressure drop (dP), regurgitation, and other conditions remotely, frequently, and usually without great expense or hindrance to the patient's health. In some examples, all four chambers of the heart and surrounding vessels may be measured to monitor and treat multiple diseases. Monitoring and measuring flow and other characteristics in peripheral parts of the body may also allow diagnostics and improved treatment for peripheral vascular diseases and diabetes.

In many cases, the periphery is accessed during surgical procedures and vascular closure devices are used to stop bleeding and facilitate healing of punctures and incisions. Patients thus accessed may benefit from various types of diagnostic sensor implantation, but currently available sensor technology is limited to implantation at locations away from the periphery, such as in the pulmonary artery. Many such devices also have limited means for precisely securing the sensor in place in the artery or other structure where they are implanted. There is therefore a need for improvements in vascular sensor implantation technologies.

### SUMMARY

One aspect of the present disclosure relates to a sensor implantation assembly, which may comprise a tissue puncture closure device. The closure device may comprise a proximal end portion and a distal end portion, a suture extending from the proximal end portion to the distal end portion of the closure device, a suture anchor assembly configured to be inserted through a tissue wall puncture, and a sealing pad positionable around the suture at the distal end portion of the closure device. The suture assembly anchor may be attached to the suture at the distal end portion of the closure device, and may comprise a diagnostic sensor.

In this sensor implantation assembly the suture may be non-biologically resorbable and may be attached to the diagnostic sensor. The suture anchor assembly may comprise an anchor, wherein the anchor is attached to the diagnostic sensor. This anchor and the sealing pad may be biologically resorbable. A second diagnostic sensor ay be connected to the suture proximal to the sealing pad. The diagnostic sensor may comprise a pressure sensor or a microelectromechanical system (MEMS) device. The diagnostic sensors may also be wirelessly readable.

The suture anchor assembly may further comprise a sensor anchor, wherein the diagnostic sensor is attached to the suture anchor. The sensor anchor may comprise a secondary suture extending through the tissue wall. The suture may wrap around or extend through the diagnostic sensor.

In another embodiment, a sensor implantation assembly for depositing a diagnostic sensor in a body of a patient is described. The assembly may comprise a tissue puncture closure device, which includes a proximal end portion and a distal end portion. A suture may extend from the proximal end portion to the distal end portion of the closure device. A suture anchor may be attached to the suture at the distal end portion of the closure device, with the suture anchor being configured to be inserted through a tissue wall puncture. A sealing pad may be positioned around the suture proximal to the suture anchor, with the sealing pad being configured to seal the tissue wall puncture upon advancement of the sealing pad toward the suture anchor. A diagnostic sensor may also be included that is connected to the suture of the tissue puncture closure device. The diagnostic sensor may be positioned proximal to the sealing pad along the suture and may be configured to sense at least one property of the body of the patient.

In this assembly, a sensor anchor may be included that is configured to attach the diagnostic sensor to a tissue wall in which the tissue wall puncture is formed. The sensor anchor may be configured to extend at least partially peripherally around a tissue wall. The sensor anchor may be configured to apply pressure to the tissue wall. The sensor anchor may comprise a suture configured to extend through a tissue wall.

The tissue puncture closure device may comprise a biologically resorbable material. In some embodiments, the suture anchor and the sealing pad comprise a biologically resorbable material and the suture comprises a non-resorbable material. The diagnostic sensor may be slidable along the suture.

Another aspect of the disclosure relates to a method of positioning a diagnostic sensor within a tissue wall through an incision. The method comprises providing a tissue puncture closure device including a suture, a suture anchor assembly, and a sealing pad, with the suture anchor assembly being attached to a distal end portion of the suture and with the suture anchor assembly comprising a diagnostic sensor. The method also includes inserting the suture anchor assembly through an incision in a tissue wall to a position within the tissue wall, seating the suture anchor assembly against an inner surface of the tissue wall with the suture anchor assembly resisting withdrawal of the suture anchor assembly through the incision, and deploying the sealing pad along the suture, wherein the sealing pad seals the incision.

In this method, deploying the sealing pad along the suture may comprise compacting the sealing pad in the incision. The method may also include positioning at least one secondary suture through the tissue wall, with the at least one secondary suture attaching the diagnostic sensor to the tissue wall independent of the suture of the tissue puncture closure device. Data may be collected from the diagnostic sensor wirelessly through the tissue wall. The diagnostic sensor may remain secured to the tissue wall after biological resorption of the suture and sealing pad.

The suture anchor assembly may be inserted through the incision within a carrier tube, wherein the suture anchor assembly may have a longitudinal axis that is substantially parallel with a longitudinal axis of the carrier tube while the suture anchor assembly is within the carrier tube. The suture anchor assembly may be rotated upon insertion through the incision.

Yet another aspect of the disclosure relates to a method of positioning a diagnostic sensor within a tissue wall through an incision, wherein the method includes providing a tissue puncture closure device including a suture, a suture anchor, and a sealing pad, with the suture anchor being attached to a distal end portion of the suture. The method may also include inserting the suture anchor through the incision to a position within the tissue wall, with the suture anchor resisting withdrawal of the suture anchor through the incision. The method may also comprise deploying the sealing pad along the suture, with the sealing pad sealing the incision, and positioning a diagnostic sensor at a position proximal to the sealing pad, with the diagnostic sensor being attached to the suture of the tissue puncture closure device.

In some arrangements the method includes deploying a sensor anchor to secure the diagnostic sensor to the tissue wall independent of the suture of the tissue puncture closure device. Deploying the sensor anchor may comprises deploying a clip around the diagnostic sensor, connecting at least one secondary suture to the diagnostic sensor and to the tissue wall, and/or securing the diagnostic sensor to the tissue wall after biological resorption of the tissue puncture closure device.

This method may also comprise wirelessly collecting data from the diagnostic sensor.

The above summary of the present invention is not intended to describe each embodiment or every implementation of the present invention. The Figures and the detailed description that follow more particularly exemplify preferred embodiments.

### BRIEF DESCRIPTION OF THE DRAWINGS

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIG. 1 is a partial cut-away view of a tissue closure device according to the prior art.

FIG. 2 is a side view of the tissue closure device of FIG. 1 engaged with an artery according to the prior art.

FIG. 3 is a side view of the tissue closure device of FIG. 1 being withdrawn from an artery according to the prior art to deploy a collagen sponge.

FIG. 4 is a side view of the tissue closure device of FIG. 1 illustrating tamping of the collagen sponge according to the prior art.

FIG. 5A is a perspective view of a micro electromechanical diagnostic sensor according to the prior art.

FIG. 5B is a section view of the diagnostic sensor of FIG. 5A taken through section lines 5B-5B in FIG. 5A.

FIG. 6A is a side view of a tissue closure device according to the present disclosure

FIG. 6B is a side section view of the distal end of the device of FIG. 6A.

FIG. 6C is a side view of the device of FIG. 6A inserted into a tissue puncture.

FIG. 6D is a section view of the distal end of the device of FIG. 6C.

FIG. 6E is a side view of the device of FIG. 6A inserted into a tissue puncture as the device is retracted.

FIG. 6F is a section view of the distal end of the device of FIG. 6E.

FIG. 6G is a side view of the device of FIG. 6A inserted into a tissue puncture as the device is being retracted.

FIG. 6H is a section view of the distal end of the device of FIG. 6G.

FIG. 6I is a section view of a tissue puncture after closure by the device of FIG. 6A.

FIG. 6J is a section view of the tissue puncture of FIG. 6I after biological resorption of the sealing pad.

FIG. 7 is a section view of another embodiment of a layered anchor and a sensor of a closure device according to the present disclosure.

FIG. 8 is a section view of another embodiment of an anchor of a closure device according to the present disclosure wherein the anchor comprises a sensor.

FIG. 9 is a section view of another embodiment of an anchor of a closure device according to the present disclosure wherein the anchor comprises a sensor.

FIG. 10A shows a section view of a tissue puncture after closure by a closure device wherein the sensor is external to the puncture.

FIG. 10B shows a section view of the closure device of FIG. 10A wherein the sealing pad has biologically resorbed.

FIG. 10C shows another embodiment of the closure device of FIG. 10A wherein the anchor has biologically resorbed.

FIG. 11A shows a section view of another embodiment of a closure device having secondary sutures.

FIG. 11B shows the closure device of FIG. 11A wherein the anchor, sealing pad, and closure suture have biologically resorbed.

FIG. 12A is a perspective view of an embodiment of sensor implantation assembly wherein the sensor is attached to the exterior of a vessel.

FIG. 12B is a section view of the device and vessel of FIG. 12A.

FIG. 13A is a side section view of a distal end of a closure device having a sensor according to another embodiment of the present disclosure.

FIG. 13B is a side view of the closure device of FIG. 13A wherein the distal end of the device has been inserted into a lumen in a vessel.

FIG. 13C is a perspective view of the distal end of the closure device of FIG. 13B within the lumen.

FIG. 13D is a side view of the closure device of FIG. 13A at another position relative to the lumen.

FIG. 13E is a perspective view of the distal end of the closure device and vessel of FIG. 13D.

FIG. 13F is a perspective view of the distal end of the closure device and vessel of FIG. 13A at another position.

FIG. 13G is a side view of the distal end of the closure device and vessel of FIG. 13A at a position sealing the interior and exterior of the puncture in the vessel.

FIG. 14 is a side view of another embodiment of a closure device and vessel, wherein the sensor is mounted external to the vessel puncture.

FIG. 15 is a side view of another embodiment of a closure device and vessel, wherein a sensor is mounted internal and external to the vessel puncture.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure generally relates to systems and methods for implantation of sensors at the site of an incision or puncture through a tissue wall such as a blood vessel wall in a human body. As mentioned above, vascular procedures often require access to an artery through a puncture. Most often, the artery is a femoral artery. To close the puncture following completion of the procedure, many times a closure device is used to sandwich the puncture between an anchor and a sealing pad. The present disclosure describes methods and apparatus that facilitate positioning a diagnostic sensor at the anchoring location of the closure device or sealing pad at the situs of the arteriotomy.

While the vascular instruments shown and described below include procedure sheaths and puncture sealing devices, the application of principles described herein are not limited to the specific devices shown. The principles described herein may be used with any medical device. Therefore, while the description below is directed primarily ₜo arterial procedures and certain embodiments of a vascular closure device, the methods and apparatus are only limited by the appended claims.

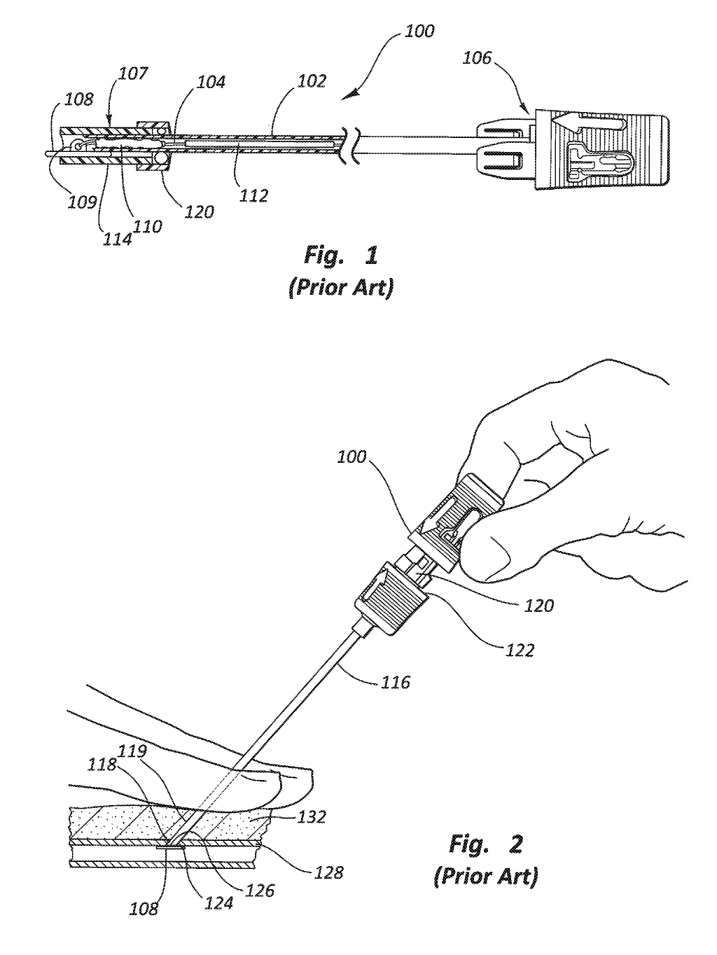

Referring now to the drawings, and in particular to FIGS. 1-4, a vascular puncture closure device 100 is shown according to the prior art. The vascular puncture closure device 100 includes a carrier tube 102 with a filament or suture 104 extending at least partially therethrough. The closure device 100 also includes a first or proximal end 106 and a second or distal end 107. External to a second or distal end 107 of the carrier tube 102 is an anchor 108. The anchor is an elongated, stiff, low profile member including an eye 109 formed at the middle. The anchor 108 is typically made of a biologically resorbable polymer.

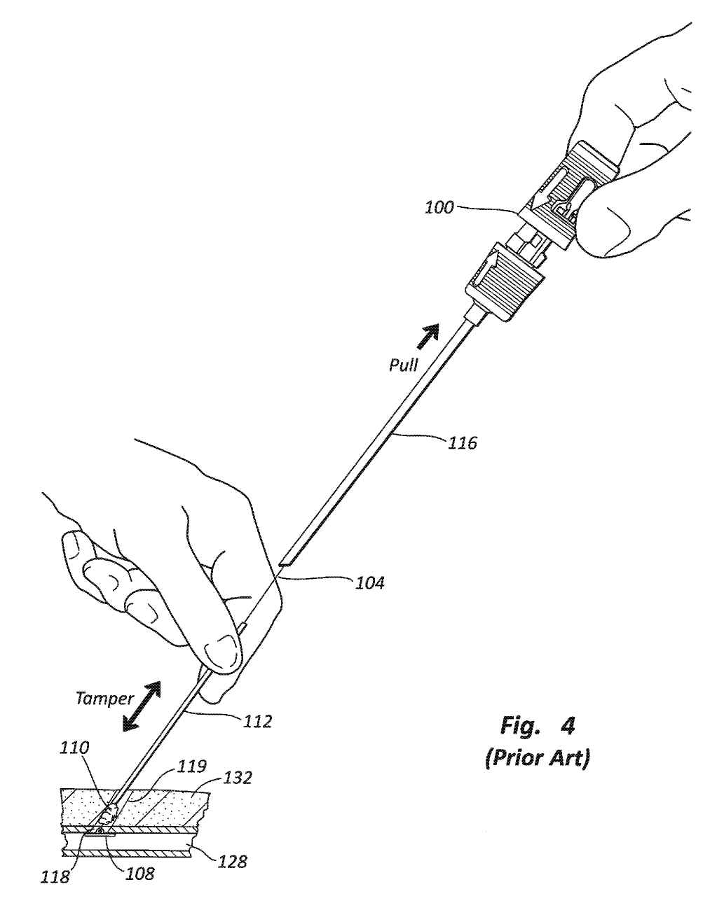

The suture 104 is threaded through the anchor 108 and back to a collagen pad 110. The collagen pad 110 may be comprised of randomly oriented fibrous material bound together by chemical means. The collagen pad 110 is slidingly attached to the suture 104 as the suture passes distally through the carrier tube 102, but as the suture traverses the anchor 108 and reenters the carrier tube 102, it is securely slip knotted proximal to the collagen pad 110 to facilitate cinching of the collagen pad 110 when the closure device 100 is properly placed and the anchor 108 deployed (see FIG. 4). The carrier tube 102 typically includes a tamping tube 112 disposed therein. The tamping tube 112 is slidingly mounted on the suture 104 and may be used by an operator to tamp the collagen pad 110 toward the anchor 108 at an appropriate time to seal a tissue puncture.

Prior to deployment of the anchor 108 within an artery, the eye 109 of the anchor 108 rests outside the distal end 107 of the carrier tube 102. The anchor 108 may be temporarily held in place flush with the carrier tube 102 by a bypass tube 114 disposed over the distal end 107 of the carrier tube 102.

The flush arrangement of the anchor 108 and carrier tube 102 allows the anchor 108 to be inserted into a procedure sheath such as insertion sheath 116 as shown in FIGS. 2-4, and eventually through a tissue puncture 118. The insertion sheath 116 is shown in FIGS. 2-4 inserted through a percutaneous incision tract 119 and into an artery 128. However, the bypass tube 114 (FIG. 1) includes an oversized head 120 that prevents the bypass tube 114 from passing through an internal passage of the insertion sheath 116. Therefore, as the puncture closure device 100 is inserted into the insertion sheath 116, the oversized head 120 bears against a surface 122 of insertion sheath 116. Further insertion of the puncture closure device 100 results in sliding movement between the carrier tube 102 (FIG. 1) and the bypass tube 114, releasing the anchor 108 from the bypass tube 114 (FIG. 1). However, the anchor 108 remains in the flush arrangement shown in FIG. 1 following release from the bypass tube 114, limited in movement by the insertion sheath 116.

The insertion sheath 116 includes a monofold 124 at a second or distal end 126 thereof. The monofold 124 acts as a one-way valve to the anchor 108. The monofold 124 is plastically deformed in a portion of the insertion sheath 116 that elastically flexes as the anchor 108 is pushed out through the distal end 126 of the insertion sheath 116. Typically, after the anchor 108 passes through the distal end 126 of the insertion sheath 116 and enters the artery 128, the anchor 108 is no longer constrained to the flush arrangement with respect to the carrier tube 102 and it deploys and rotates to the position shown in FIG. 2.

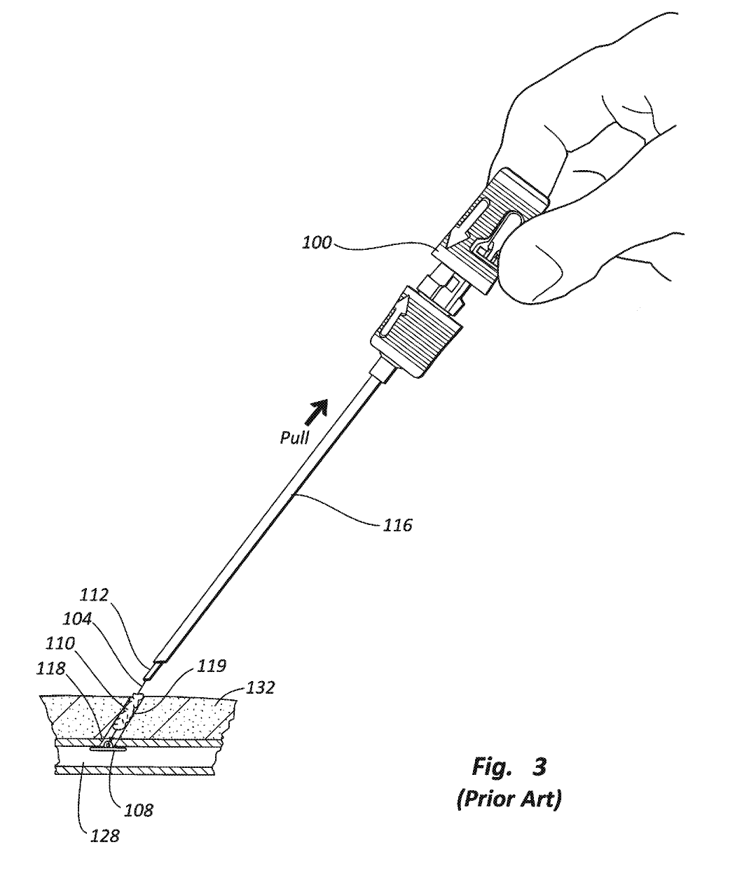

Referring next to FIGS. 3-4, with the anchor 108 deployed, the puncture closure device 100 and the insertion sheath 116 are withdrawn together, ejecting the collagen pad 110 from the carrier tube 102 into the incision tract 119 and exposing the tamping tube 112. With the tamping tube 112 fully exposed as shown in FIG. 4, the collagen pad 110 is manually tamped, and the anchor 108 and collagen pad 110 are cinched together and held in place with the self-tightening slip-knot on the suture 104. Thus, the tissue puncture 118 is sandwiched between the anchor 108 and the collagen pad 110, thereby sealing the tissue puncture 118. The suture 104 is then cut and the incision tract 119 may be closed. The suture 104, anchor 108, and collagen pad 110 are generally made of entirely resorbable materials and therefore remain in place while the puncture 118 heals.

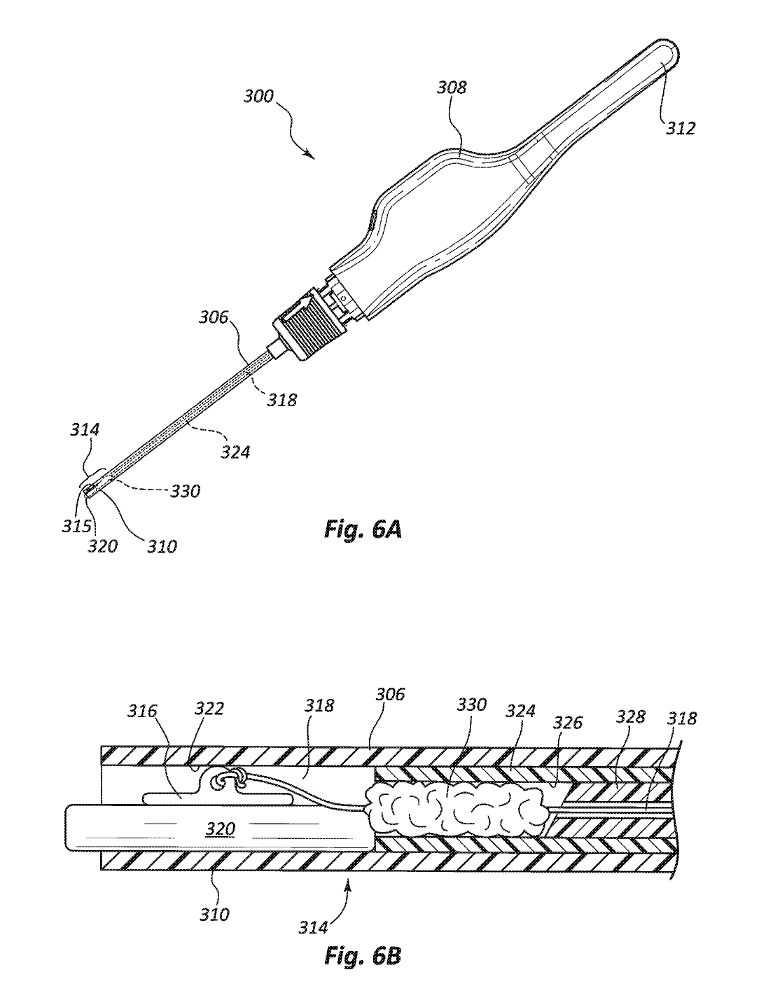

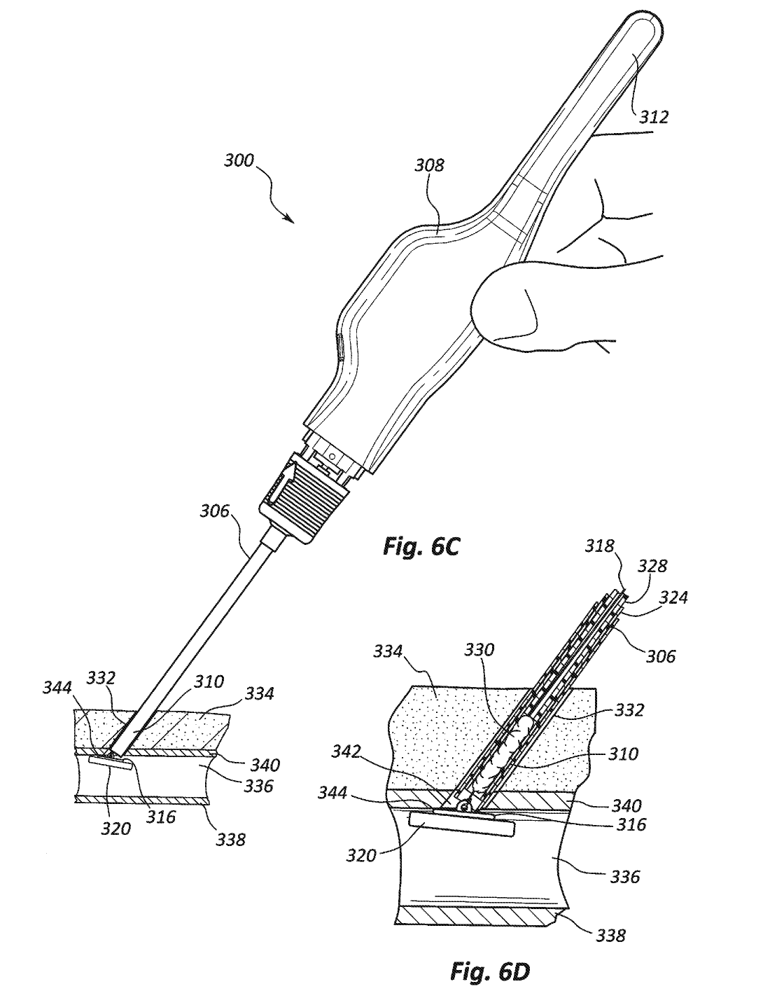

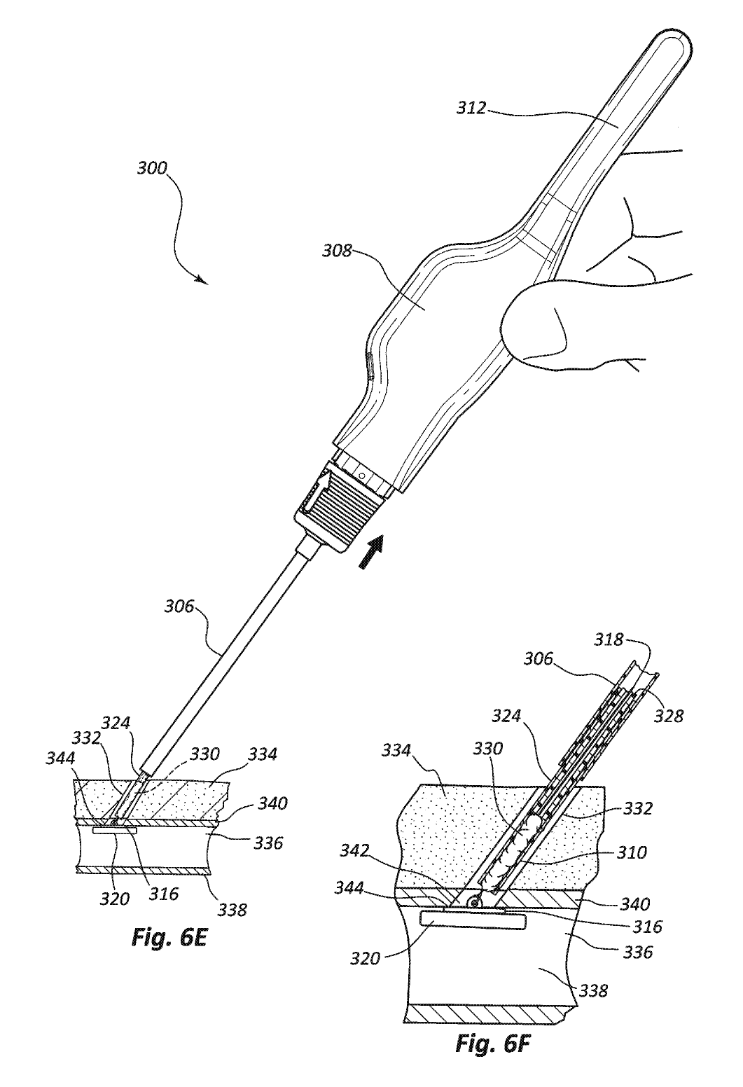

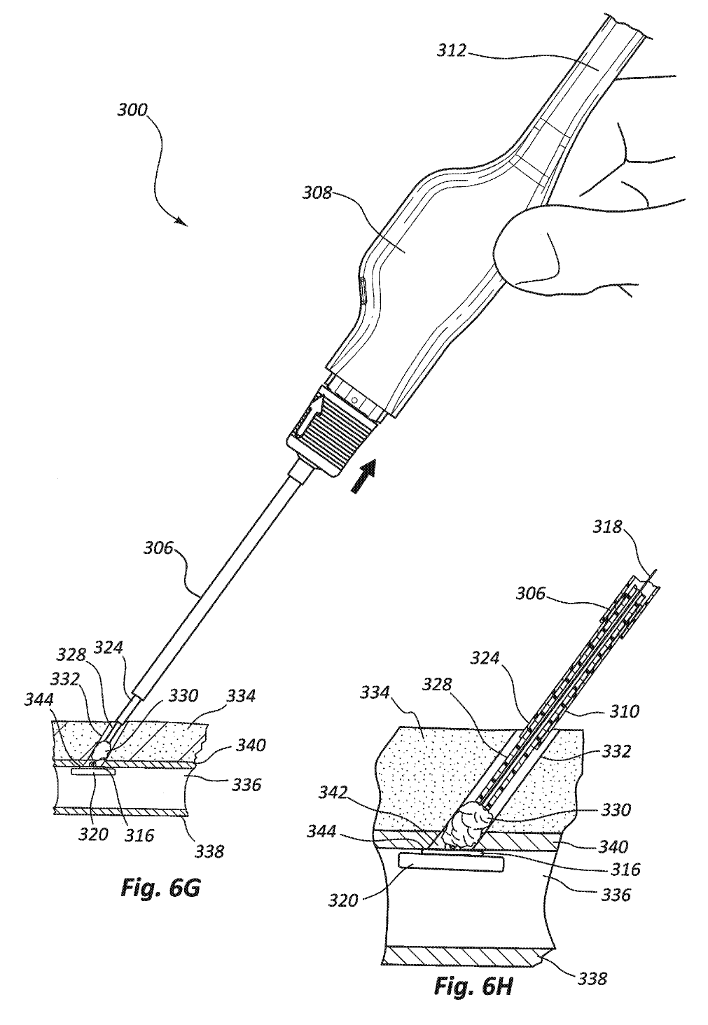

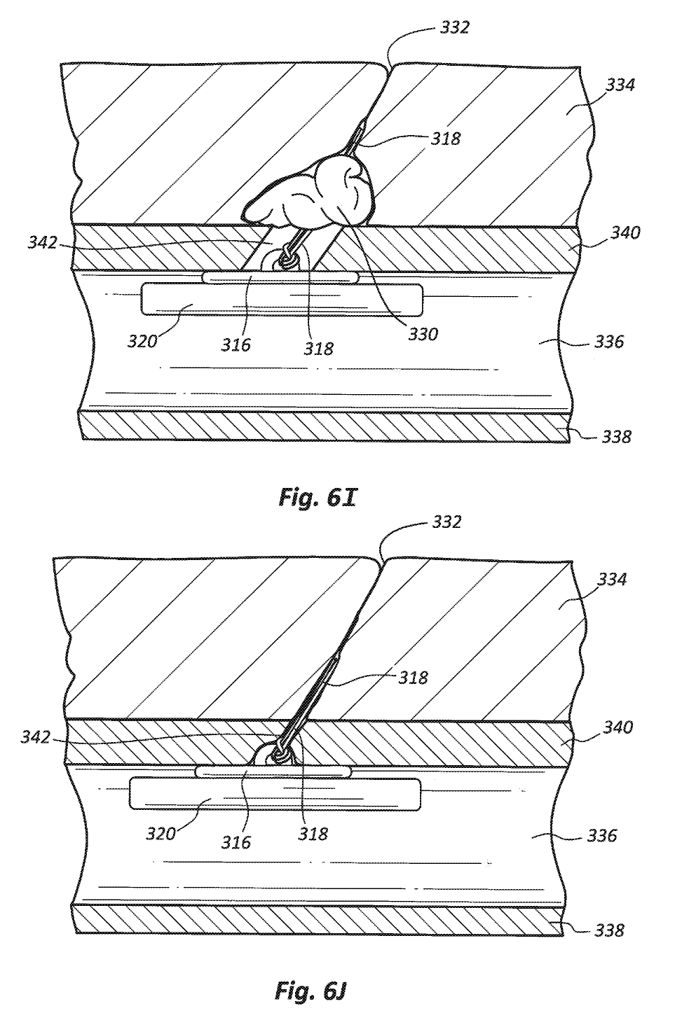

Various other closure devices exist in the prior art, including automatic tamping closure devices wherein a mechanism in a handle of the closure device advances a tamping tube 112 through an insertion sheath 116. An example automatic tamping tissue closure device is described in detail in U.S. Pat. No. 7,837,705, issued 23 Nov. 2010, which is hereby incorporated by reference in the present application in its entirety. An automatic tamping tissue closure device may tamp the collagen pad 110 using a compaction tube upon withdrawal of a handle of the device. An example embodiment of a tissue closure tamping device is described in connection with FIGS. 6A-6J below.

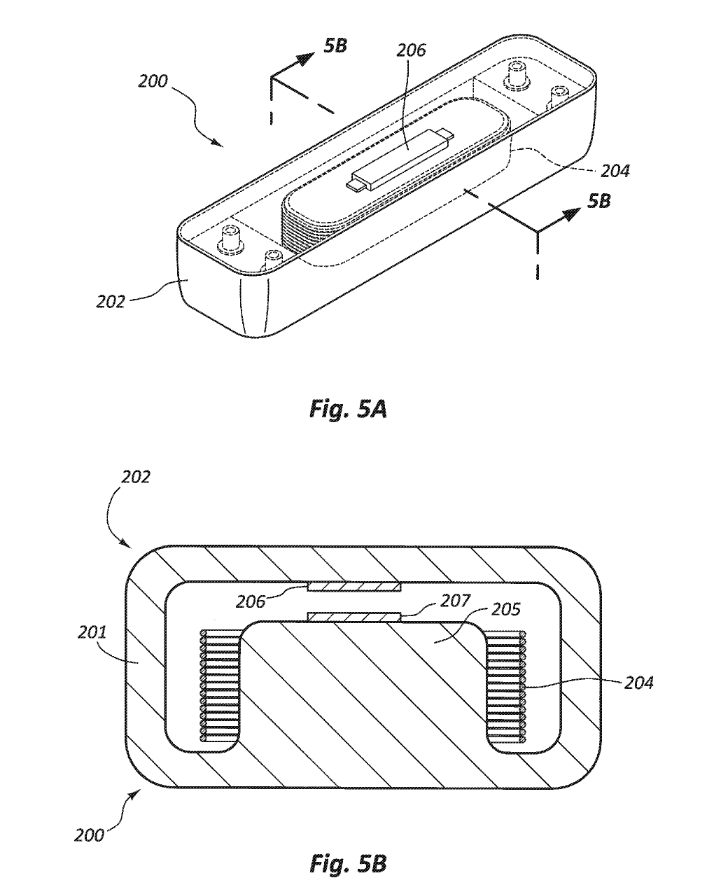

FIGS. 5A and 5B show views of a cardiovascular diagnostic sensor according to the prior art. Sensor 200 generally includes a body 202 formed of a generally hollow fused silica housing 201 with a silicone coating around the exterior of the housing. An elongated boss 205, also formed from fused silica, may project into the interior of housing 201 and may be formed integrally therewith. A plurality of electrically conductive windings may wrap around boss 205 to form an inductor coil 204. Capacitive plates 206 and 207 are separated by micrometer spacing, forming a variable capacitor. Capacitive plate 206 is sensitive to pressure and experiences nanometer scale deflections due to changes in blood pressure acting on the sensor 200. These nanometer scale deflections result in a change in the resonant frequency of the circuit formed by the inductor coil 204 and pressure-sensitive capacitor formed by plates 206 and 207. The Resonant Frequency f_{R}=1/(2π*sqrt(L×C(p))), where L is the inductance of inductor coil 204 and C(p) is the capacitance which varies with pressure. The entire assembly is hermetically sealed and does not come in contact with blood.

The sensor 200 can be electromagnetically coupled to a transmitting antenna (not shown). Consequently, a current is induced in the sensor 200, which oscillates at the resonant frequency of the circuit formed by the inductor coil 204 and pressure-sensitive capacitor formed by plates 206 and 207. This oscillation causes a change in the frequency spectrum of the transmitted signal. From this change, the bandwidth and resonant frequency of the particular sensor may be determined, from which the corresponding blood pressure can be calculated. Time-resolved blood pressure measurements can be correlated to flow and other relevant diagnostic metrics using empirical relationships established in clinical literature.

In some embodiments, sensor 200 may include optional nitinol loops extending from each end of body 202 to stabilize the sensor at an implant location. It will be appreciated that sensor 200 includes no additional leads, batteries or active-fixation mechanisms. Sensor 200 is an externally modulated inductor-capacitor circuit, which is powered using radio frequency by the transmitting antenna. Additionally, sensor 200 may be relatively small (e.g., 3.5×2×15 mm). Other advantages of sensor 200 include its accuracy, durability, biocompatibility, and insensitivity to changes in body chemistry, biology or external pressure. Sensor 200 may optionally include one or more radiopaque components to aid in localization and imaging of the device.

Sensor 200 may be modified for various applications and tuned to selectively emphasize different parameters. For example, by varying the width of the windings of inductor coil 204, the number of turns and the gap between the upper and lower windings, the resonant frequency that the device operates at and the pressure sensitivity (i.e., the change in frequency as a result of deflection of the pressure sensitive capacitive plate 206) can be optimized for different applications. In general, the design allows for a very small gap between the windings (typically between about 3 and about 35 microns) that in turn provides a high degree of sensitivity while requiring only a nanometer scale movement of the capacitive plates 206 and 207 to sense pressure changes.

The thickness of sensor 200 may also be varied to alter mechanical properties. Thicker substrates for forming housing 201 are more durable for manufacturing. Thinner substrates allow for creation of thin pressure sensitive membranes for added sensitivity. In order to optimize both properties, sensor 200 may be manufactured using two complementary substrates of different thicknesses. For example, one side of sensor 200 may be constructed from a substrate having a thickness of about 200 microns. This provides the ability to develop and tune sensors based on the operational environment of the implanted sensor 200. In addition to changes to housing 201, other modifications may be made to the sensor depending on the application. For example, nitinol loops or suture holes may be used for attachment, and cantilevers or other structural members may be added. In some variations, sensors may be powered by kinetic motion, the body's heat pump, glucose, electron flow, Quantum Dot Energy, and similar techniques.

Sensors 200 may be used to measure and/or calculate one or more parameters including real time blood pressure, flow velocity (e.g., blood flow), apposition forces based on pressure changes due to interaction between two surfaces of a prosthetic valve, impingement forces, which are correlated to pressure changes caused by the interaction between a surface of a prosthetic device and native tissue, cardiac output, effective orifice area, pressure drop, and aortic regurgitation. Sensor 200 provides time-resolved pressure data which may be correlated to the parameters of interest based on empirical correlations that have been presented in literature. In some examples, sensors 200 may function similar to piezoelectric strain gauges to directly measure a parameter. Other parameters may be indirectly calculated. Methods of using sensors 200 to measure aortic regurgitation or other body conditions are presented in the present disclosure with reference to FIGS. 6A through 15.

FIGS. 6A through 6J illustrate an embodiment of a tissue wall puncture closure device 300 configured to implant a sensor 320 internal to a tissue wall 340. The closure device 300 may have particular utility in connection with intravascular procedures, such as angiographic dye injection, cardiac catheterization, balloon angioplasty and other types of recanalizing of atherosclerotic arteries, etc., as the closure device 300 is designed to cause immediate hemostasis of a blood vessel puncture. However, it will be understood that while the description of the preferred embodiments below are directed to the sealing off of percutaneous punctures in arteries, such devices have much more widespread applications and can be used for sealing punctures or incisions in other types of tissue walls as well, generally in other areas where a diagnostic sensor 320 may be used to measure and collect characteristics of the body. Thus, the sealing of a percutaneous puncture in an artery, as shown herein, is merely illustrative of one particular use of the closure device 300 of the present disclosure.

FIG. 6A shows a side view of the closure device 300 prior to insertion into the body. The closure device 300 may comprise an insertion sheath 306 connected to a housing or handle 308. The insertion sheath 306 may extend to the distal end portion 310 of the device 300, and the handle 308 may be positioned at the proximal end portion 312 of the device 300. The insertion sheath 306 may contain a tissue closure assembly 314 at the distal end portion 310, as shown in detail in the cross-sectional view of FIG. 6B, which may be deployed through the opening at the distal end portion 310. A tissue closure assembly may alternatively be referred to as a tissue closure device or a sensor implantation assembly due to its ability to close a tissue puncture and to implant a sensor at or near a tissue puncture.

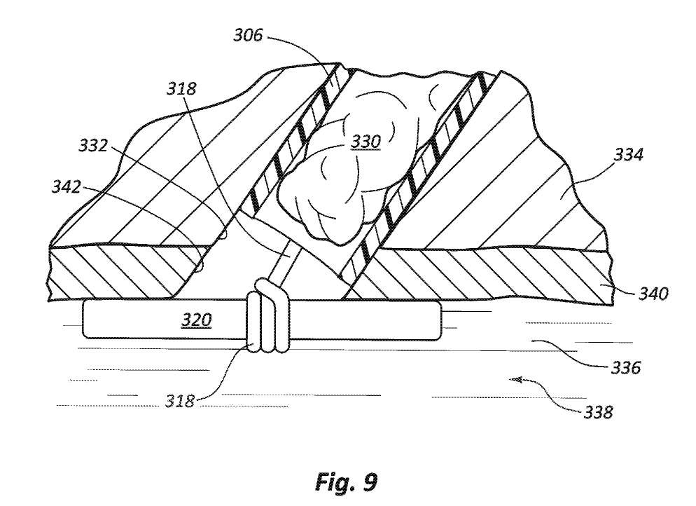

Referring to FIG. 6B, the tissue closure assembly 314 may comprise an anchor 316 connected to a distal end of a suture 318 which extends through the insertion sheath 306 to the handle 308. The anchor 316 may also be connected to a diagnostic sensor 320 that may be disposed in the insertion sheath 306 along with the anchor 316 at the time of operation of the closure device 300. The anchor 316 and sensor 320 may collectively be referred to as a suture anchor assembly. In some embodiments, the sensor 320 may be referred to as a suture anchor assembly by itself, such as in embodiments that lack a separate anchor element 316. See e.g., FIGS. 8-9.

The insertion sheath 306 may comprise an insertion sheath lumen 322 in which the anchor 316, suture 318, and sensor 320 may all be contained at the distal end portion 310 of the device 300. The insertion sheath lumen 322 may also contain two other tubular members, such as a carrier tube 324 (having a carrier tube lumen 326) and a tamping tube 328 (which is within the carrier tube lumen 326). The suture 318 may extend from the anchor 316 proximally through the carrier tube lumen 326 and through the tamping tube 328 to a spool or other suture retaining device (not shown) at the proximal end portion 312 of the device 300. A sealing pad 330 may be positioned around the suture 318 between the handle 308 and the anchor 316, and more particularly between the anchor 316 and the distal end of the tamping tube 328 at least partially within the carrier tube lumen 326. The sealing pad 330 is shown uncompressed in FIG. 6B. In some embodiments, the sealing pad 330 may comprise a flowable sealing material that is deliverable from the closure device 300.

The anchor 316, the sealing pad 330, and the suture 318 may be collectively referred to as the “closure elements” herein. As shown in FIG. 6A, the anchor 316 may be positioned adjacent to and exterior to the distal end portion 310 of the carrier tube 324, while the sealing pad 330 (FIG. 6B) is initially disposed within the carrier tube 324. The anchor 316 is shown nested in its low profile configuration along the carrier tube 324 to facilitate insertion into a body lumen in FIGS. 6A-6B, and deployed with a first surface 344 abutting the artery wall 340 of body lumen 336 in. FIGS. 6C-6I. The suture 318, non-resorbable in certain embodiments, extends distally from the proximal end portion 312 of the closure device 300 through the carrier tube 324. The suture 318 may be threaded through one or more perforations in the sealing pad 330, through a hole in the anchor 316, and proximally back toward the carrier tube 324 to the sealing pad 330. The suture 318 is preferably threaded again through a perforation or series of perforations in the sealing pad 330. The suture 318 may also be threaded around itself to form a self-tightening slip-knot. The suture 318 may thus connect the anchor 316 and the sealing pad 330 in a pulley-like arrangement to cinch the anchor 316 and the sealing pad 330 together when the carrier tube 324 is pulled away from the anchor 316 and the sealing pad 330. The anchor 316 and the sealing pad 330 sandwich and lock the anchor and plug together, sealing the vessel puncture 342. See also FIG. 6I.

The carrier tube 324 may be made of plastic or other material and is designed for insertion through the insertion sheath 306. The insertion sheath 306 is designed for insertion through a percutaneous incision 332 in a tissue layer 334 and into a lumen 336. According to FIGS. 6C-6J, the lumen 336 comprises an interior portion of a femoral artery 338.

The anchor 316 may be an elongated, stiff, low-profile member arranged to be seated inside the artery 338 against an artery wall 340 contiguous with a puncture 342. The anchor 316 is preferably made of a biologically resorbable polymer.

The sealing pad 330 is formed of a compressible sponge, foam, or fibrous mat made of a hemostatic biologically resorbable material such as collagen, and may be configured in any shape so as to facilitate sealing the vessel puncture 342. In some embodiments, the sealing pad 330 may be embodied by a flowable sealing material that may be advanced into the puncture and then may melt or otherwise flow to fill and seal the puncture. In some embodiments, the sealing pad 330 may comprise a flowable material that may be injected into the puncture and then harden to form a seal.

FIGS. 6C and 6D show the closure device 300 with the insertion sheath 306 in a first position inserted through the incision 332 in the tissue layer 334 with the anchor 316 and sensor 320 ejected from the distal end portion 310 of the insertion sheath 306. The anchor 316 and sensor 320 have been rotated from their position that is longitudinally parallel to the insertion sheath 306 in FIGS. 6A and 6B to an anchoring position out of the sheath 306 that is not parallel to the sheath 306 in FIGS. 6C and 6D. Preferably, the anchoring position has the anchor about parallel to a longitudinal axis of the artery 338 or parallel to the surface of the lumen in the artery 338 where the puncture 342 is located. The width of the anchor 316 may be sufficient to span the width of the puncture 342 at the artery wall 340 so that the anchor 316 contacts the inner surface of the artery wall 340 on each side of the puncture 342 (as shown in FIG. 6D) and, due to its rotated position that is parallel to the longitudinal axis of the lumen 336, provides resistance to withdrawal of the anchor 316 back through the puncture 342.

The carrier tube 324 may also comprise a slit 315 at the distal end portion 310 of the device 300. In some embodiments, the slit 315 may be a slot, wherein the sides of the slot are spaced apart. The slit 315 may allow opposing sides of the carrier tube 324 at the slit 315 to separate from each other. In some embodiments, this means that the distal end portion 310 of the carrier tube 324 may expand radially outward to receive an anchor 316 and sensor 320 that have a combined thickness greater than the inner diameter of the distal end portion 310 of the carrier tube 324. The insertion sheath 306 may also comprise a slit to accommodate the anchor 316 and sensor 320. In some cases, the slit in the carrier tube 324 or insertion sheath 306 does not expand radially outward when simply holding the anchor 316 and sensor 320, but the slit 315 may still relieve pressure on the components within the distal end portion 310 (e.g., the anchor 316 and sensor 320), thereby enabling easier ejection of those components at the appropriate time while still keeping them aligned with the longitudinal axis of the carrier tube 324 until the time of deployment. A slit 315 in the carrier tube 324 may be prevented from opening or flexing when surrounded by the insertion sheath 306, which is movably positioned external to and concentric with the carrier tube 324. Retraction of the handle 308 and insertion sheath 306 may cause the insertion sheath 306 to retract with respect to the carrier tube 324 to a second position shown in FIGS. 6E and 6F, in which case the slit 315 may be exposed and may therefore allow the carrier tube 324 to be radially expandable.

Upon proximal withdrawal of the handle 308 and insertion sheath 306 (and/or simultaneous distal advancement of the carrier tube 324), the carrier tube 324 may be exposed at the distal end portion 310 of the device 300, as shown in FIGS. 6E-6F. The anchor 316 and sensor 320 may be positioned fully within the artery 338. The automatic tamping assembly of the closure device 300 may then tamp down and compact the sealing pad 330 by proximal withdrawal of the carrier tube 324 and distal advancement of the tamping tube 328, as illustrated in FIGS. 6G-6H. Tension may be applied to the suture 318 as well, causing the suture 318 to tighten the sealing pad 330 such as by tightening a slip knot in the suture 318 in which the sealing pad 330 is tied or looped. This causes the sealing pad 330 to compact, fold, or compress toward the anchor 316 and sensor 320 and may sandwich the artery wall 340 in place between the sealing pad 330 and the anchor 316. Additional detail about an example automatic tamping system is described in U.S. Pat. No. 7,837,705 (which is also referenced above).

Upon completing compaction of the sealing pad 330, the handle 308 may be further proximally withdrawn to expose the suture 318, which may be cut below the external surface of the tissue layer 334 or below the skin of the patient. After the suture is cut, the anchor 316, suture 318, sensor 320, and sealing pad 330 (i.e., the tissue closure assembly 314) may remain deposited and implanted in the tissue layer 334 and/or artery 338, as shown in FIG. 6I.

The tissue closure assembly 314 may remain in the position of FIG. 6I to facilitate hemostasis and healing until resorption of the anchor 316, suture 318, and sealing pad 330. Thus, the sensor 320 may be non-bioresorbable and may remain in the artery 338 after resorption of the other components in the tissue closure assembly 314. The sensor 320 may remain within the artery 338 and be accessed wirelessly to provide diagnostic measurements through the tissue layer 334.

Various methods and/or structures may be employed to retain the sensor 338 affixed to the interior of the artery wall 340. For example, the sensor 320 may comprise external structures or features such as the nitinol loops described above. In some embodiments, the sensor 320 may comprise one or more prongs, hooks, spurs, barbs, or the like, or any other suitable attachment method or structure known to those skilled in the art, to retain or affix the sensor 320 to the inside of the artery wall 340. Alternatively, retractable structures, such as prongs, hooks, spurs, fingers, barbs, or the like, may be used. In a retracted position, such structures may allow the sensor to be deployed. When subsequently extended, such structures will retain the sensor 320 in a fixed position relative to the artery wall 340. Alternatively or in combination with such structures, tissue growth may assist to secure the sensor 320 in place on the artery wall. In other embodiments, the anchor 316 and suture 318 may be non-bioresorbable such that they remain attached to the sensor 320 after resorption of the sealing pad 330 and healing of the tissue layer 334 and artery wall 340 around them. FIG. 6J shows an example embodiment where the suture 318 remains deposited in the tissue layer 334 and artery wall 340 after resorption of the sealing pad 330. Thus, the position of the sensor 320 may be maintained by a non-resorbable anchor 316 and suture 318 after resorption of the sealing pad 330.

The final position of the sensor 320 may coincide with the situs of the puncture 342 in the artery wall 340. The ability to thereby precisely position the sensor 320 may be advantageous in ensuring that the sensor 320 is able to precisely measure appropriate diagnostic metrics at specific portions of the body that are optimally measured from a certain area in the patient's physiology. Positioning the sensor 320 at the situs of the puncture 342 also reduces or eliminates a need for a separate procedure to implant the sensor 320 in the body when a vascular access incision is formed for other purposes. For example, after conducting a surgery requiring vascular access, the same puncture that would already have to be closed using a vascular closure device can also be used to implant the sensor 320. This is particularly beneficial for implantation of the sensor 320 to obtain diagnostic information in the periphery of the body where catheter-based surgical tools are often already used through punctures to perform other procedures.

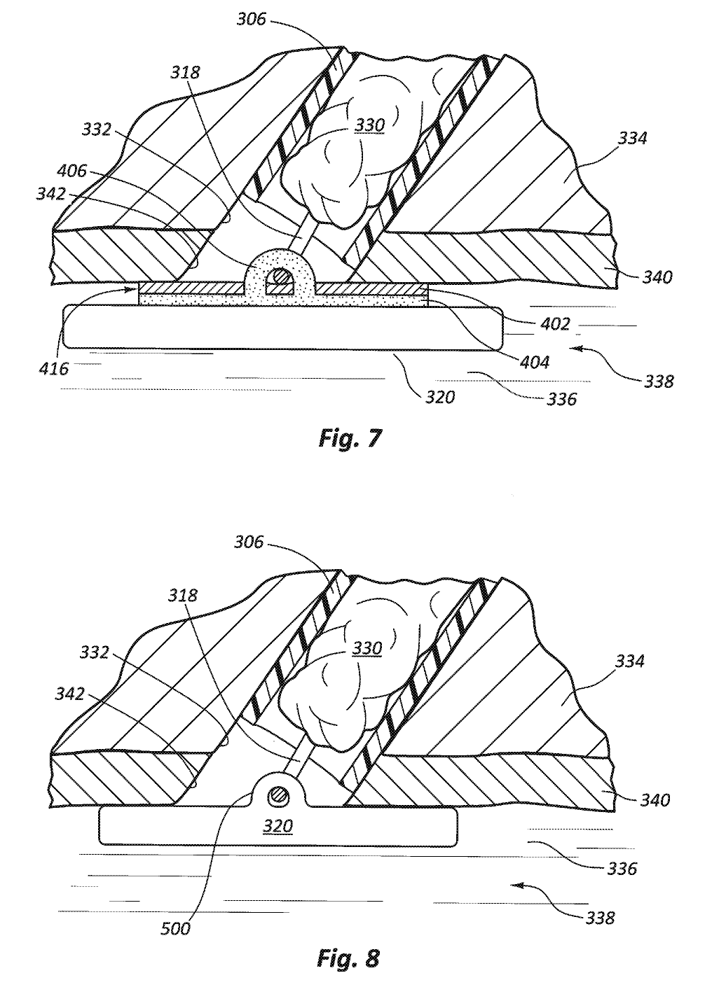

A plurality of other systems may be implemented to secure the position of the sensor 320 in the artery 338 after closure of the puncture 342. FIG. 7 shows an embodiment including a layered anchor 416 to which a sensor 320 may be attached. The layered anchor 416 may comprise at least two materials arranged in layers. A first layer 402 may be positioned on a side of the anchor 416 facing away from (i.e., proximally relative to) the sensor 320 when the anchor 416 is positioned in an artery 338. The side of the anchor 416 contacting or facing the sensor 320 may comprise a second layer 404. The first and second layers 402, 404 may comprise materials that biologically resorb at different rates or that comprise materials wherein the first layer 402 resorbs over time, but the second layer 404 does not resorb.

In configurations where the first and second layers 402, 404 of anchor 416 resorb at different rates, the first layer 402 may resorb before the second layer 404. This may allow the anchor 416 to reduce its thickness more quickly on one side of the anchor 416 as the puncture 342 heals and may therefore reduce the duration of occlusion of flow in the artery 338 by the sensor 320 and anchor 416 more quickly than an anchor (e.g., 316) with a uniform resorption rate. In order for the layers to resorb at different rates, the layers may, for example, be formed of different materials or may comprise a different surface texture, wherein the first layer 402 may have more exposed surface area as compared to the second layer 404 and may thus dissolve and resorb more quickly than the second layer 404.

The layered anchor 416 may also comprise an attachment feature 406 for connecting the anchor 416 to the suture 318. FIG. 7 shows that the attachment feature 406 may be an eyelet or other aperture through which the suture 318 may extend to keep the suture 318 attached to the anchor 416. The attachment feature 406 may comprise the material of the second layer 404 in order to preserve the attachment feature 406 and to prevent it from resorbing before the first layer 402.

In another embodiment, the anchor 416 may comprise a plurality of apertures through the anchor 416 (e.g., through both layers 402, 404) and/or sensor 320 instead of, or in addition to, the eyelet, and the apertures may be the attachment feature. A suture 318 may extend through the apertures to secure the anchor 416 and/or sensor 320 in place.

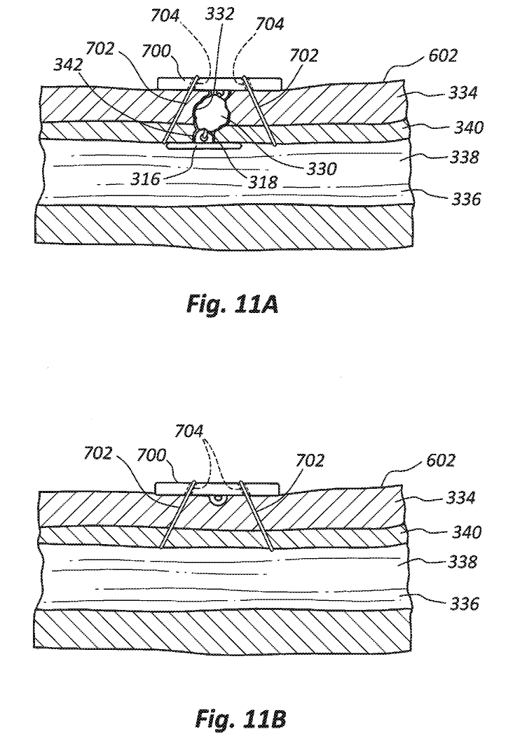

FIG. 8 shows an alternative embodiment wherein no separate anchor 316 is implemented. Instead, the sensor 320 comprises an attachment feature 500 that is connected to the suture 318 without an intervening anchor. The sensor 320 may therefore be used as an anchor in place of anchor 316 during implantation of the sensor 320 (e.g., in the steps shown in FIGS. 6C to 6H). Alternatively, the anchor 316 may be integrally formed with the sensor 320 as a suture anchor assembly. The attachment feature 500 of the sensor 320 may be an eyelet, as shown in FIG. 8, or may comprise one or more apertures forming a passage through the sensor 320 that the suture 318 may extend through. Apertures of this nature are discussed in further detail in connection with FIGS. 11A-11B.

FIG. 9 shows an example embodiment wherein the suture 318 extends around or is tied to the sensor 320. For example, the suture 318 may be tied around the sensor 320 while the sensor 320 is used as an anchor. In some embodiments, the sensor 320 may have an external groove or protrusion. The suture 318 may be seated in the groove or may be held in position around the sensor by the protrusion in order to prevent the sensor 320 from being inadvertently detached from the suture 318.

In the embodiments of FIGS. 8-9, the sensor 320 is not resorbed, and the suture 318 may be used to retain the sensor 320 in position in the artery 338 after resorption of the sealing pad 330. Thus, these embodiments may appear similar to the embodiment of FIG. 6J, but the sensor 320 being positioned alone in place of the assembly of the anchor 316 and sensor 320.

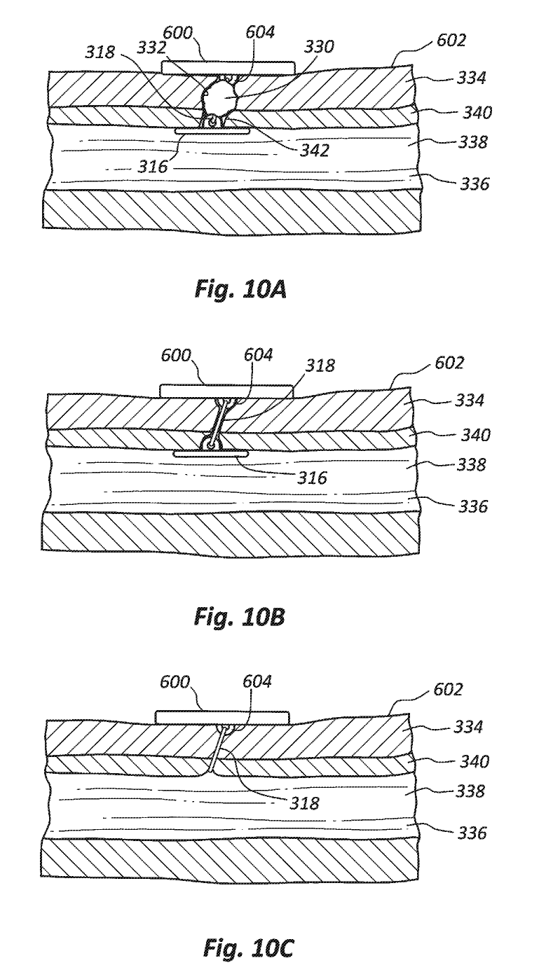

The closure device 300 of FIGS. 6A-6H may also be used in conjunction with a system and method for implantation of a sensor at the exterior surface of a tissue wall. For instance, in some embodiments, the closure device 300 may not have a sensor 320 attached to or positioned with the anchor 316 in a suture anchor assembly, and the closure device 300 may close a puncture by leaving only an anchor 316, suture 318, and sealing pad 330 behind. FIGS. 10A-11B illustrate systems that allow a sensor to be attached to the exterior of a vessel wall during or after the closure of a vessel puncture using a closure device 300.

FIG. 10A shows an embodiment where the incision 332 and puncture 342 have been closed using a closure device (e.g., closure device 300) without a sensor (e.g., 320) being implanted within the artery 338. In this embodiment, the sensor 600 is attached to the suture 318 at a position external to the tissue wall 334, such as on an external surface 602 of the tissue wall 334. The sensor 600 may have an eyelet 604, aperture, or other connection feature to allow the sensor 600 to stay attached to the suture 318 and anchor 316 as the incision 332 and puncture 342 heal. For example, the sensor 600 may be threaded onto and attached to the suture 318 before or after the suture 318 is cut in an otherwise typical tissue closure procedure. In some arrangements, the sensor 600 may be tightened toward the anchor 316 when it is attached to the suture 318 in order to apply pressure to the tissue wall 334 and thereby facilitate hemostasis at the incision 332 while securing the sensor 600 in place.

As shown in FIG. 10B, after healing of the incision 332 and puncture 342 and after resorption of the sealing pad 330, the sensor 600 may remain held in place at the external surface 602 of the tissue wall 334 by the suture 318 and anchor 316 which have not resorbed. The sensor 600 may therefore remain in position at the external surface 602 to measure diagnostic information over time.

In another embodiment, shown in FIG. 10C, the anchor 316 may biologically resorb after implantation of the sensor 600, leaving the sensor 600 at the external surface 602 secured by the suture 318 alone. As shown in FIGS. 10B and 10C, the tissue closure assembly used may partially resorb (e.g., just the sealing pad 330 or the sealing pad 330 and the anchor 316 may resorb) to leave behind a non-resorbable attachment structure, such as, for example, the suture 318 and/or anchor 316 that keeps the sensor 600 properly positioned. The non-resorbable suture may be the same suture 318 used to close the vessel puncture 342. In some embodiments, a secondary suture may be used to secure the sensor 600 in place as well. The secondary suture may be a suture that is not used as part of the closure device.

FIGS. 11A and 11B show another configuration for implantation of a diagnostic sensor 700 at the site of a closure device. The sensor 700 may be implanted at the outer surface 602 of the tissue wall 334 in the manner shown in FIG. 10A, but in this case, the sensor 700 may be further secured by a secondary attachment feature, such as secondary sutures 702 that extend through the tissue wall 334 in addition to the suture 318 used in connection with the closure device 300. Thus, the sensor 700 may be attached to a plurality of sutures 318, 702. The secondary sutures 702 may be attached to the sensor 700 and the tissue wall 334 after closure of the puncture 342 or as an additional part of the closure process.

The secondary sutures 702 may be non-bioresorbable and the closure suture 318 may be bioresorbable. As shown in FIG. 11B, the closure suture 318, anchor 316, and sealing pad 330 may resorb, thereby leaving the sensor 700 and secondary sutures 702 implanted in the body afterward. The secondary sutures 702 may comprise one or more loops of suture extending through the tissue wall 334 and into the artery 338, as shown in the two segments of the secondary sutures 702 of FIGS. 11A-11B. In some embodiments, the secondary sutures 702 may be tied partially into or through the tissue wall 334 and/or artery 338 instead of extending completely therethrough. In another embodiment, there may only be one secondary suture 702 and/or the suture 702 may not form a loop, but may have an inner anchor (e.g., a knot) that keeps the suture 702 secured to the tissue wall 334.

The sensor 700 may be configured with apertures 704 through which the secondary sutures 702 may extend. Alternatively, the secondary sutures 702 may wrap around the exterior of the sensor 700, such as, for example, around its body or within a groove or other surface feature configured to help retain the connection between the sensor 700 and the secondary sutures 702 by resisting longitudinal movement of the sensor 700.

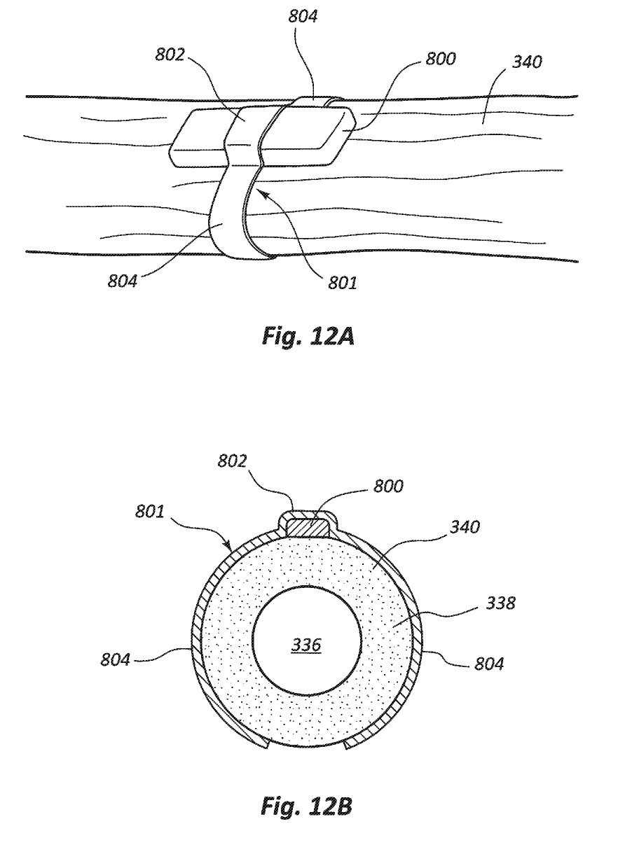

In the embodiments shown in. FIGS. 10A-11B, the sensors 600, 700 are shown positioned external to a tissue wall 334 and external to an artery 338. The position of the sensors 600, 700 may alternatively be on the surface of the artery wall 340, as shown by sensor 800 in FIG. 12A. The type of sensor used may also influence the orientation of the sensor before and after implantation. For example, one sensor may have a length axis configured to be positioned parallel to a lumen in which it is planted, and another sensor may require orientation perpendicular to the lumen in which it is planted. Thus, a sensor may be rotated or otherwise positioned upon entry into the lumen (or other body cavity) to ensure preferable orientation.

FIGS. 12A-12B show an embodiment wherein a clip 801 or other external attachment device may be used to secure a sensor 800 to the exterior of an artery wall 340. In this embodiment, the sensor 800 may be initially attached to the artery 338 using a closure device described elsewhere herein (e.g., closure device 300), and the sensor 800 may be retained at least temporarily to the artery wall 340 by a closure suture 318, such as in the manner shown in FIG. 10A. After attachment of the sensor 800 using the closure suture 318, a clip 801 or external attachment device may be introduced to the situs of the sensor 800 to attach the sensor 800 to the artery 338. FIGS. 12A-12B show an example of a clip 801 that comprises a midsection 802 and two peripheral arms 804. The midsection 802 may be formed to fit around or connect to the sensor 800, and the arms 804 may be configured to extend away from the midsection 802 and extend around the artery wall 340 to retain the sensor 800 in place. The midsection 802 in this embodiment forms a band which covers a partial length of the sensor 800 (see FIG. 12A) and that follows the outer perimeter of the sensor 800 on three sides (see FIG. 12B). In some embodiments, the arms 804 may be configured to flex radially inward relative to the artery 338 to allow the clip 801 to grip the artery 338 by a pressure fit. Thus, the clip 801 may keep the sensor 800 in place on the artery 338 after resorption of an anchor 316, suture 318, and sealing pad 330. FIG. 12B shows a section view of the artery 338 after resorption of those closure elements.

Using a clip 801 to retain the sensor 800 may eliminate a need to use non-bioresorbable suture to keep the sensor 800 in place after resorption of other elements of the tissue closure assembly 314. Thus, the artery 338 is not penetrated by the sensor 800 or its retaining device (i.e., sensor anchor) after healing and resorption of the closure assembly. The sensor 800 may also be easier to remove at a later time, if necessary, since no suture would need to be cut or removed from the artery 338 at that time.

While the above-described embodiments specifically disclose closure devices and associated methods that implant one sensor in the body, it will be appreciated that in some cases a plurality of sensors may be implanted using these devices and methods. For example, in one embodiment a first sensor may be implanted internal to the artery wall 340 (i.e., in the lumen 336 of the artery 338, as shown in FIG. 6J) and a second sensor may be implanted external to the artery wall 340 (e.g., in the position shown in FIG. 10C) using the same suture (e.g., 318).

In another example embodiment, multiple sensors 800 may be implanted circumferentially or peripherally around an artery 338 using a clip configured to hold a plurality of sensors against the artery wall 340. Thus, multiple types of sensors may be implanted in multiple locations within, without, and around a tissue wall 334 or artery wall 340. This may allow different kinds of measurements or may provide redundancy to the sensor systems, as needed.

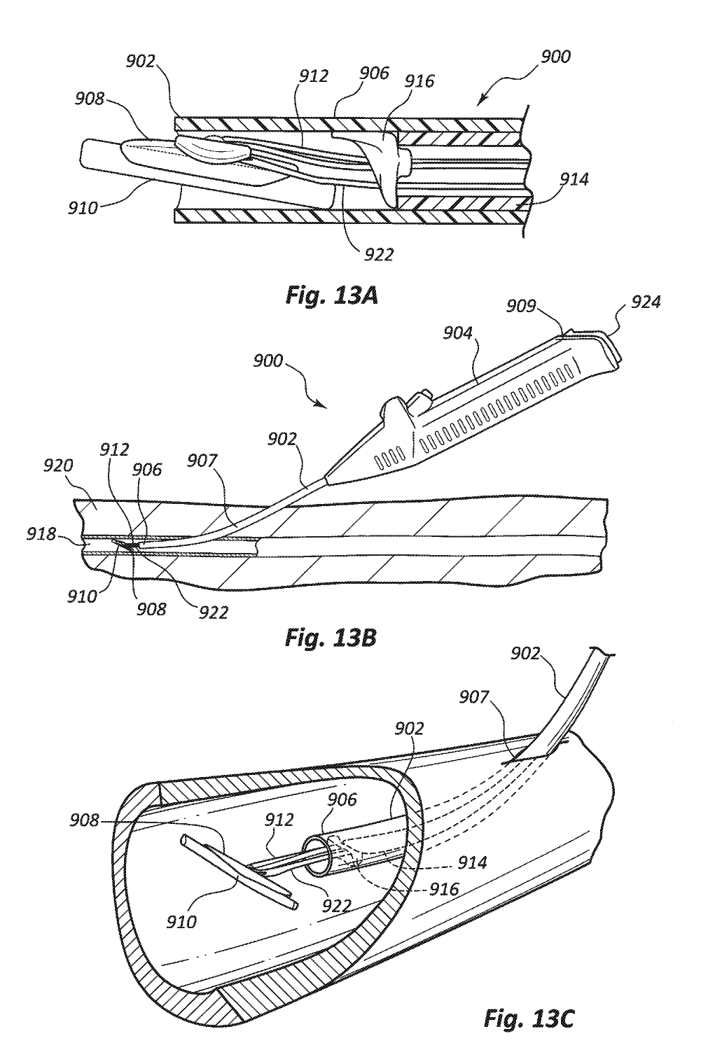

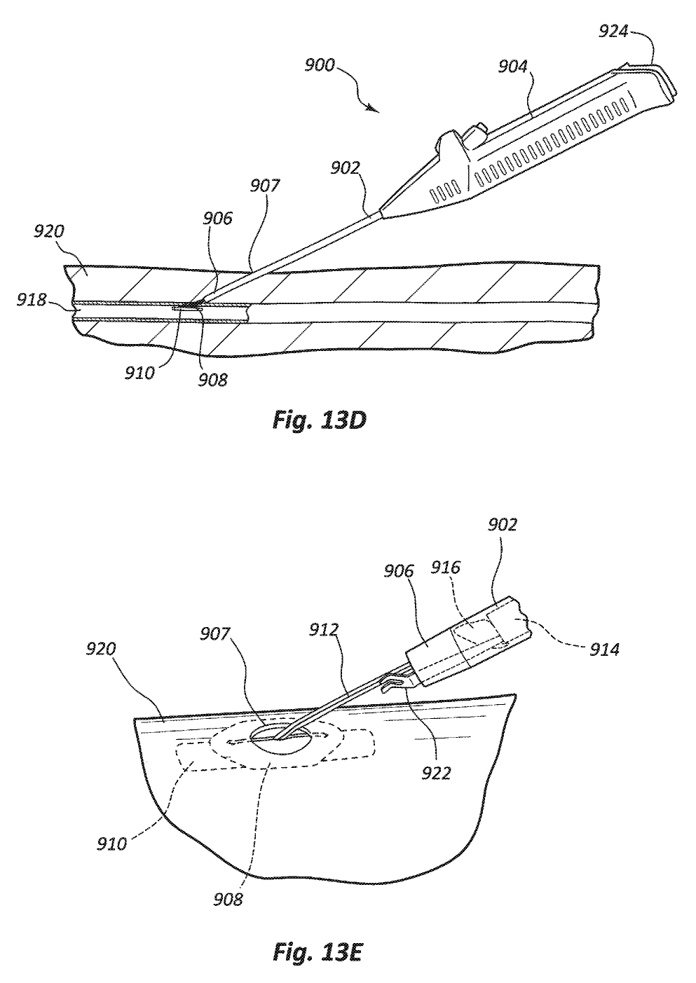

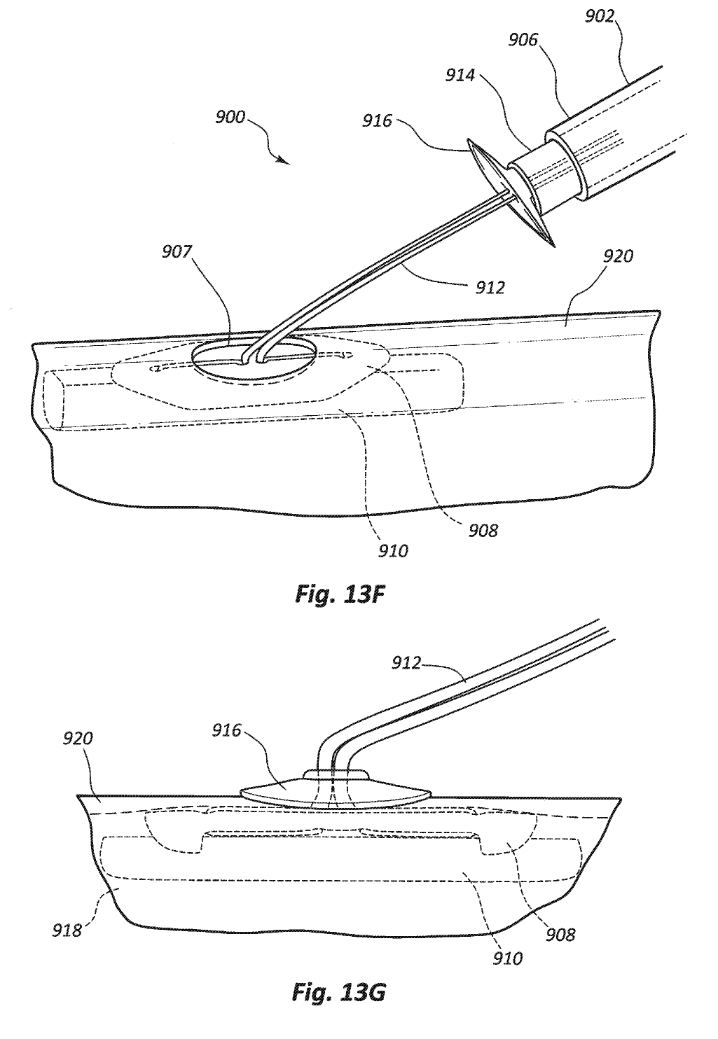

FIGS. 13A-13G show yet another embodiment of a closure device 900 having sensor implantation ability. The closure device 900 may have an insertion sheath 902 extending from a handle 904. The distal end portion 906 of the closure device 900 is insertable into a tissue puncture 907 while a proximal end portion 909 remains external to the puncture 907. See FIG. 13B.

Referring now to FIG. 13A, before insertion into the tissue puncture 907, the distal end portion 906 may contain an inner seal member 908 attached to a sensor 910 in the insertion sheath 902. A suture 912 may be connected to the inner seal member 908 in a manner similar to the connection between the anchor 316 and suture 318 of embodiments described above. The suture 912 may extend proximally through the insertion sheath 902 and a delivery tube 914 (which is internal to the insertion sheath 902) into a connection point or spool (not shown) in the handle 904. An outer seal member 916 may also be threaded onto the suture 912 proximal to the inner seal member 908. The outer seal member 916 may also be positioned distal to the delivery tube 914 and within the insertion sheath 902.

FIGS. 13B-13G illustrate the steps taken to implant the sensor 910 while closing the tissue puncture 907 using the closure device 900. In FIGS. 13B-13C, the insertion sheath 902 is inserted through the tissue puncture 907 and into a cavity or lumen 918 beneath a layer of tissue 920. The lumen 918 may be an arterial lumen similar to the artery 338 described above. In some embodiments, the lumen 918 may be an opening within a body organ or other fluid or gas-filled opening beneath a tissue wall.

Upon introduction of the insertion sheath 902, the inner seal member 908 and sensor 910 may be ejected from the insertion sheath 902 using an ejection member 922. The ejection member 922 may contact the inner seal member 908 on a proximal side of the inner seal member 908 and extend proximally into the handle 904. A button 924 or other control feature on the handle 904 may be used to advance the ejection member 922 distally, thereby urging the inner seal member 908 and sensor 910 out of the distal end portion 906 of the closure device 900. Upon exiting the distal end portion 906, the inner seal member 908 may reorient itself from a first position substantially aligned with a longitudinal axis of the insertion sheath 902 to a second position rotated from the first position toward a more perpendicular orientation with respect o the longitudinal axis of the sheath 902. See FIGS. 13A and 13C.

Next, as shown in FIGS. 13D-13E, the inner seal member 908 may be withdrawn proximally until it is oriented to achieve hemostasis at the wall of the lumen 918 where the puncture 907 is located. The inner seal member 908 may therefore be used as a seal for the puncture 907 and as an anchor for the suture. The ejection member 922 may retract proximally away from the inner seal member 908. Thus, the inner seal member 908 may abut the opening of the puncture 907 and seal the puncture 907 with the sensor 910 positioned distal of the inner seal member 908 relative to the insertion sheath 902. The sensor 910 is also positioned internal to the inner seal member 908 relative to the lumen 918.

FIG. 13F illustrates the ejection of the outer seal member 916 from the insertion sheath 902. The closure device 900 may be operated to advance the delivery tube 914 distally relative to the insertion sheath 902, thereby pushing the outer seal member 916 out of the insertion sheath 902. The outer seal member 916 may be partially compacted or longitudinally oriented while it is positioned within the insertion sheath 902 (as shown in FIG. 13A), but after ejection from the insertion sheath 902 by the delivery tube 914 it may radially expand to the shape shown in FIG. 13F.

After ejection from the insertion sheath 902, the outer seal member 916 may be advanced along the suture 912 toward the inner seal member 908 and sensor 910 until it fits against the tissue layer 920 external to the puncture 907, as shown in FIG. 13G. Thus, the tissue layer 920 may be sandwiched between the inner seal member 908 and the outer seal member 916. The suture 912 may then be cut off proximal to the outer seal member 916 and the closure device 900 may be removed. The suture 912 may be tied off or otherwise tightened against the outer seal member 916 before or after cutting to keep the outer seal member 916 pressed against the tissue layer 920 at least for the duration of time needed for the puncture 907 to heal.

The inner and outer seal members 908, 916 and suture 912 may or may not be biologically resorbable. The sensor 910 therefore may remain connected to a non-resorbable suture 912 via the inner seal member 908 and/or outer seal member 916 being non-resorbable as well, or the suture 912 may be non-resorbable while the seal members 908, 916 are resorbable. The suture 912 may be directly connected to the sensor 910 or may only be connected to the inner seal member 908. Thus, in various embodiments, the sensor 910 may be retained to the interior of the tissue layer 920 in a manner similar to the various embodiments shown in FIGS. 6J, 7, 8, and 9, but with an inner seal member 908 instead of an anchor (e.g., 316, 416) and outer seal member 916 instead of a sealing pad (e.g., 330). For example, the sensor 910 may have an eyelet comparable to the eyelet 500 of the sensor 320 shown in FIG. 8. In some cases, secondary sutures extending through the tissue layer 920 may be used to secure the sensor 910 within the tissue layer 920, similar to the secondary sutures of FIG. 11B.

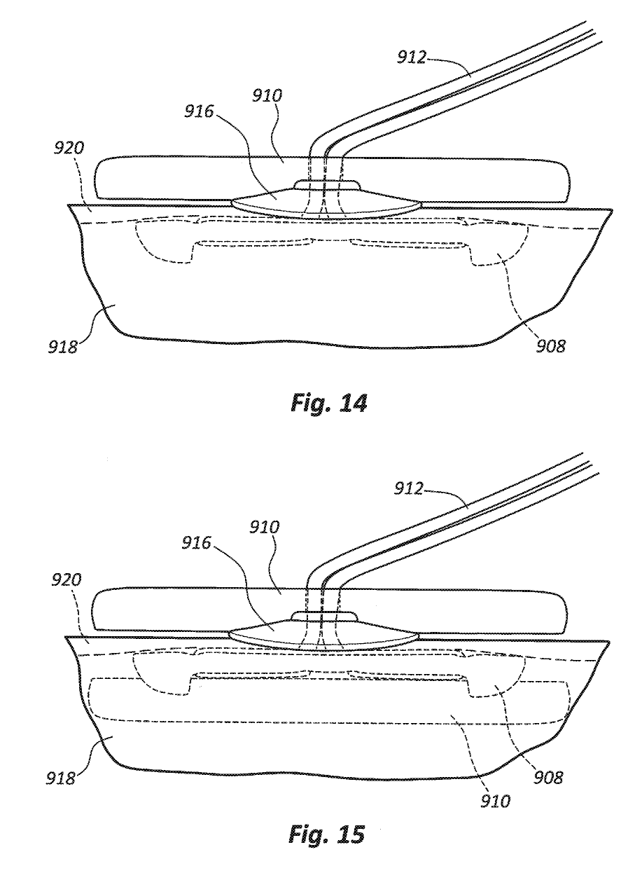

In another embodiment, shown in FIG. 14, the closure device 900 may be operated without the sensor 910 being attached to the inner seal member 908 but instead being attached to the outer seal member 916. Generally, the sensor 910 is attached to the proximal side of the outer seal member 916 and is advanced to the tissue layer 920 by the delivery tube 914 along with the outer seal member 916. The sensor 910 may therefore be disposed on the interior or exterior of the tissue layer 920. The sensor 910 may be retained to the tissue layer 920 using various systems disclosed elsewhere herein (however using the seal members 908, 916), such as the systems shown in FIGS. 10A-12B.

In yet another embodiment, the closure device 900 may have a first sensor attached to the inner seal member 908 and a second sensor attached to the outer seal member 916, such that the inner and outer seal members 908, 916 may each have a separate sensor. Alternatively, each seal member 908, 916 may be attached to a separate part of the same sensor device. For example, the inner seal member 908 may be attached to a sensor and the outer seal member 916 may be attached to an antenna used with the sensor on the inner seal member 908. FIG. 15 shows an example embodiment wherein two sensors 910 are attached to opposite sides of the tissue layer 920 by being attached to separate seal members 908, 916.

The present description provides examples, and is not limiting of the scope, applicability, or configuration set forth in the claims. Thus, it will be understood that changes may be made in the function and arrangement of elements discussed without departing from the spirit and scope of the disclosure, and various embodiments may omit, substitute, or add other procedures or components as appropriate. For instance, the methods described may be performed in an order different from that described, and various steps may be added, omitted, or combined. Also, features described with respect to certain embodiments may be combined in other embodiments.

Various inventions have been described herein with reference to certain specific embodiments and examples. However, they will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including:” and “having” come as used in the specification and claims shall have the same meaning as the term “comprising.”

What is claimed is:

1. A sensor implantation assembly, comprising:
a tissue puncture closure device, comprising:
a proximal end portion and a distal end portion;
a suture extending from the proximal end portion to the distal end portion of the closure device;
a suture anchor assembly configured to be inserted through a tissue wall puncture, the suture anchor assembly being attached to the suture at the distal end portion of the closure device, the suture anchor assembly comprising a diagnostic sensor;
a sealing pad positionable around the suture at the distal end portion of the closure device.

2. The sensor implantation assembly of claim 1, wherein the suture is non-biologically resorbable.

3. The sensor implantation assembly of claim 2, wherein the suture is attached to the diagnostic sensor of the suture anchor assembly.

4. The sensor implantation assembly of claim 1, wherein the suture anchor assembly comprises an anchor, the anchor being attached to the diagnostic sensor.

5. The sensor implantation assembly of claim 4, wherein the anchor and the sealing pad are biologically resorbable.

6. The sensor implantation assembly of claim 1, further comprising a second diagnostic sensor connected to the suture proximal to the sealing pad.

7. The sensor implantation assembly of claim 1, wherein the diagnostic sensor comprises a pressure sensor.

8. The sensor implantation assembly of claim 1, wherein the diagnostic sensor comprises a microelectromechanical system (MEMS) device.

9. The sensor implantation assembly of claim 1, wherein the diagnostic sensor is wirelessly readable.

10. The sensor implantation assembly of claim 1, wherein the diagnostic sensor is wirelessly powered.

11. The sensor implantation assembly of claim 1, wherein the suture anchor assembly further comprises a sensor anchor, the diagnostic sensor being attached to the suture anchor.

12. The sensor implantation assembly of claim 10, wherein the sensor anchor comprises a secondary suture extending through the tissue wall.

13. The sensor implantation assembly of claim 1, wherein the suture wraps around or extends through the diagnostic sensor.

14. A sensor implantation assembly for depositing a diagnostic sensor in a body of a patient, comprising:
a tissue puncture closure device, comprising:
a proximal end portion and a distal end portion;
a suture extending from the proximal end portion to the distal end portion of the closure device;
a suture anchor attached to the suture at the distal end portion of the closure device, the suture anchor being configured to be inserted through a tissue wall puncture;
a sealing pad positioned around the suture proximal to the suture anchor, the sealing pad being configured to seal the tissue wall puncture upon advancement of the sealing pad toward the suture anchor;
a diagnostic sensor connected to the suture of the tissue puncture closure device, the diagnostic sensor being positioned proximal to the sealing pad, the diagnostic sensor being configured to sense at least one property of the body of the patient.

15. The sensor implantation assembly of claim 14, further comprising a sensor anchor configured to attach the diagnostic sensor to a tissue wall in which the tissue wall puncture is formed.

16. The sensor implantation assembly of claim 15, wherein the sensor anchor is configured to extend at least partially peripherally around a tissue wall.

17. The sensor implantation assembly of claim 15, wherein the sensor anchor is configured to apply pressure to the tissue wall.

18. The sensor implantation assembly of claim 15, wherein the sensor anchor comprises a suture configured to extend through a tissue wall.

19. The sensor implantation assembly of claim 14, wherein the tissue puncture closure device comprises a biologically resorbable material.

20. The sensor implantation assembly of claim 14, wherein the suture anchor and the sealing pad comprise a biologically resorbable material and the suture comprises a non-resorbable material.

21. The sensor implantation assembly of claim 14, wherein the diagnostic sensor is slidable along the suture.

22. A method of positioning a diagnostic sensor within a tissue wall through an incision, the method comprising:
providing a tissue puncture closure device including a suture, a suture anchor assembly, and a sealing pad, the suture anchor assembly being attached to a distal end portion of the suture, the suture anchor assembly comprising a diagnostic sensor;
inserting the suture anchor assembly through an incision in a tissue wall to a position within the tissue wall;
seating the suture anchor assembly against an inner surface of the tissue wall, the suture anchor assembly resisting withdrawal of the suture anchor assembly through the incision;
deploying the sealing pad along the suture, the sealing pad sealing the incision.

23. The method of claim 22, wherein deploying the sealing pad along the suture comprises compacting the sealing pad in the incision.

24. The method of claim 22, further comprising positioning at least one secondary suture through the tissue wall, the at least one secondary suture attaching the diagnostic sensor to the tissue wall independent of the suture of the tissue puncture closure device.

25. The method of claim 22, further comprising collecting data from the diagnostic sensor wirelessly through the tissue wall.

26. The method of claim 22, wherein the diagnostic sensor remains secured to the tissue wall after biological resorption of the suture and sealing pad.

27. The method of claim 22, wherein the suture anchor assembly is inserted through the incision within a carrier tube, the suture anchor assembly having a longitudinal axis substantially parallel with a longitudinal axis of the carrier tube while the suture anchor assembly is within the carrier tube.

28. The method of claim 27, further comprising rotating the suture anchor assembly upon insertion through the incision.

29. A method of positioning a diagnostic sensor within a tissue wall through an incision, the method comprising:
providing a tissue puncture closure device including a suture, a suture anchor, and a sealing pad, the suture anchor being attached to a distal end portion of the suture;
inserting the suture anchor through the incision to a position within the tissue wall, the suture anchor resisting withdrawal of the suture anchor through the incision;
deploying the sealing pad along the suture, the sealing pad sealing the incision;
positioning a diagnostic sensor at a position proximal to the sealing pad, the diagnostic sensor being attached to the suture of the tissue puncture closure device.

30. The method of claim 29, further comprising deploying a sensor anchor to secure the diagnostic sensor to the tissue wall independent of the suture of the tissue puncture closure device.

31. The method of claim 30, wherein deploying the sensor anchor comprises deploying a clip around the diagnostic sensor.

32. The method of claim 30, wherein deploying the sensor anchor comprises connecting at least one secondary suture to the diagnostic sensor and to the tissue wall.

33. The method of claim 30, wherein deploying the sensor anchor secures the diagnostic sensor to the tissue wall after biological resorption of the tissue puncture closure device.

34. The method of claim 29, further comprising wirelessly collecting data from the diagnostic sensor.
